(19) United States
(12) Patent Application Publication
(10) Pub. No.: US 2026/0328721 A1
(43) Pub. Date: Oct. 22, 2026

(54) TELLURENE-BASED ELECTROCHEMICAL SENSOR SYSTEM AND METHOD

(71) Applicant: Purdue Research Foundation, West Lafayette, IN (US)

(72) Inventors: Wenzhuo Wu, West Lafayette, IN (US); Ruifang Zhang, West Lafayette, IN (US)

(73) Assignee: Purdue Research Foundation, West Lafayette, IN (US)

(21) Appl. No.: 19/180,439

(22) Filed: Apr. 16, 2025

Related U.S. Application Data
(60) Provisional application No. 63/635,535, filed on Apr. 17, 2024.

(51) Int. Cl. G01N 27/327; G01N 27/30; G01N 27/40; G01N 33/94

(52) U.S. Cl. CPC G01N 27/3278; G01N 27/301; G01N 27/308; G01N 27/40; G01N 33/9413; G01N 33/9433

(57) ABSTRACT

An electrochemical sensor system includes a substrate, a working electrode, a counter electrode, a reference electrode, and an adhesive layer. The working electrode includes a tellurene-based material. The working electrode may also include a metallic nanoparticle(s) disposed on the tellurene-based material. The counter electrode may include a carbon-based material. The reference electrode may include a silver/silver chloride-based material. The adhesive layer may be permeable to sweat. The system may further include a polydimethylsiloxane (PDMS) layer that includes microfluidic channels. The working electrode, the counter electrode, the reference electrode, and the PDMS layer may be sandwiched between the substrate and the adhesive layer. The electrochemical sensor system may be configured to detect a neurotransmitter from sweat.

### CROSS REFERENCE TO RELATED APPLICATIONS

This application claims the benefit of U.S. Provisional Application No. 63/635,535 filed Apr. 17, 2024, the entirety of which is hereby incorporated by reference.

### GOVERNMENT FUNDING

This invention was made with government support under CMMI-2046936 awarded by the National Science Foundation. The government has certain rights in the invention.

### FIELD

The disclosure generally relates to sensor systems and, more particularly, to wearable electrochemical sensor systems.

### INTRODUCTION

This section provides background information related to the present disclosure which is not necessarily prior art.

The longitudinal variations of neurotransmitters (NTs) dynamics in body fluids (e.g., blood and brain fluid) are closely related to mental and physical health. NTs are chemicals that serve as messengers in synaptic transmission and are key players in brain functions. Any abnormalities or changes in neurotransmitters' activities may cause severe psychotic and neurodegenerative diseases related to human mental and physical health. Among them, monoamines (e.g., dopamine (DA), norepinephrine (NE)) and/or their metabolites are important biomarkers for the diagnosis, therapy and prognosis of several neuroendocrine and cardiovascular disorders in multiple regulatory systems and metabolic processes. Neurodegenerative disease affected 4.7 to 6.0 million individuals in the U.S. and the annual cost was $ 655 billion in 2020 USD. DA and NE are also used as critical indicators for assessing stress level. Stress disorder affected 11.8 million individuals in the U.S., and the annual economic burden to the US was estimated as $232 billion. They act via dopaminergic and adrenergic receptors, regulating the stress response, psychomotor activity, emotional processes, learning, sleep, and memory.

The capacity to measure real-time longitudinal variations of DA and NE in body fluids can enable just-in-time diagnosis and early personalized intervention to prevent progression and significantly improve treatment outcomes of many physiological and psychiatric disorders. The existence of multiple interpretations of dopaminergic and adrenergic function can be attributed to the inherent difficulties in achieving real-time visualization of DA/NE neurotransmission and its associated behavioral responses in humans. These difficulties stem from the ultrafast kinetics of neurotransmitter release, which occurs within milliseconds and presents significant technical challenges for studies. Furthermore, the limited time resolution of current techniques utilized to study these neurotransmitters exacerbates the challenge of real-time observation of neurotransmission. Real-time detection of NTs in biofluids can alert the person about the plausible onset of disease. Monitoring drug efficacy is another scenario in which continuous measurements are important. Monitoring neurotransmitter dynamics is essential to understanding the communication between neurons and their target cells and developing therapeutic neuromodulatory strategies.

However, the current approaches for measuring them in blood and brain fluid do not allow real-time longitudinal monitoring of related dynamics. Current techniques for the detection and quantification of neurotransmitters include microdialysis, Liquid chromatography with mass spectroscopy (HPLC-MS), fluorescence spectroscopy, electrophoresis, and optical methods such as photoluminescence, colorimetric analysis, etc. Another disadvantage of current techniques is that they exhibit low temporal resolution and difficulty collecting biofluid samples. Therefore, all current techniques show infeasibility for real-time longitudinal monitoring of the trend in the change of NT concentrations in the human body. Additionally, these standard techniques require lengthy procedures and specialized bulky equipment to collect and analyze sweat samples.

Sweat contains a plethora of biomarkers related to physiological and mental health. Sweat can be collected non-invasively. Wearable sweat sensors provide opportunities for continuous monitoring of evolving endogenous conditions without interfering with or interrupting normal activities. Sweat can be collected non-invasively and on-demand (such as through local chemical stimulation) at convenient locations on the body, ideal for continuously monitoring the levels of various metabolites. Sensors can be placed close to the site of sweat generation, allowing for fast detection before analytes biodegrade. The growing field of sweat-based wearable sensors aims to tackle the limitations of centralized, reactive healthcare by giving individuals insight into the dynamics of their physiology. By relaying physiological information as the body evolves over healthy and sick states, sensors will enable individuals to longitudinal monitor themselves in high temporal resolution without expensive equipment or trained professionals. The most accessible biofluid, sweat, contains a wealth of chemical information that could indicate the body's deeper biomolecular state. Most water-soluble substances dissolved in plasma appear in sweat, some in more significant and some in lower concentrations in sweat than in plasma. Some reports show the presence of NTs (DA/NE) in human sweat using HPLC-MS. However, these identified NTs in sweat were not quantitatively measured. Moreover, no wearable sweat sensors have been reported to detect DA or NE in real sweat. The longitudinal quantification of these NTs using wearable sweat sensors, which is unavailable using current technologies, would enable diagnosis and monitor various conditions, including mental health disorders, neurological conditions, and endocrine disorders.

In order to detect the presence and the amount of NTs accurately and restrict the interference to the minimum extent, numerous efforts are devoted to modifying the surface of glass carbon electrodes (GCE), which are widely used as standard working electrodes due to their low cost, broad electrochemical window, stability, to construct electrochemical biosensors with high sensitivity and specificity. However, GCEs have a relatively low surface area compared to nanomaterials, limiting the analyte sensing area. Metal nanostructures with high surface areas, such as gold, cobalt, and silver, are widely applied to modify the GCE to enhance the sensors'electrical conductivity and electrocatalytic activity. Incorporating metal nano-structured materials enables faster and more efficient binding of the analyte, leading to faster electron transfer and more efficient oxidation of analytes with enhanced redox currents and sensitivity. Another family of materials for modifying electrode surfaces is carbon-based nanostructures, such as carbon nanotubes and graphene. For instance, graphene has an extraordinary electrochemical surface area, high conductivity, and wide electrochemical potential window, making it a strong candidate for electrochemical sensing of many metabolites, including redox-silent materials. The biosensors made of GCEs and those decorated with metal nanostructures or carbon-based materials demonstrated superior conductivity and electrocatalytic activity. However, their selectivity to different analytes is limited due to similar electrocatalytic activity for specific redox reactions. For instance, in the electrochemical detection of DA, ascorbic acid (AA) and uric acid (UA) always co-exist with DA in blood, urine, and extracellular fluids. The oxidation potentials of these biomarkers are close to each other (peak difference between 0.1-0.3 V). In addition, the concentrations of UA and AA are two to three orders of magnitude higher than DA, thus making the electrochemical detection to determine the DA's amount a challenging task. Compared to DA, NE is more challenging to detect electrochemically due to its lower concentration in biological fluids and its lower stability compared to DA. In addition, the oxidation products of DA/NE can be chemisorbed on the surface of electrodes, leading to the poisoned electrodes and a narrow linear range. For example, metal nanostructure-modified electrodes are easily fouled by products after sensing. In the electrochemical detection of DA and NE, the DA and NE molecules would be oxidized under a specific voltage (0.2-0.5 V). An oxidation current would be generated as a correspondence on the working electrodes, and the magnitude of the oxidation current could help assess the concentration of the DA and NE. The accumulation of the positive charges (e.g., holes) on the electrode would lower the energy barrier of DA/NE oxidation. Thus, the oxidation current would increase, improving the sensitivity of the as-fabricated biosensors. According to the surface adsorption theory, catalysis is based on the adsorption/desorption of reactant molecules on the catalyst's surface, as during this process, an electron transfer between the absorbed molecules and the catalyst surface would happen. Therefore, p-type semiconductors are promising for the electrochemical detection of DA/NE. In addition, another requirement raised for the catalyst is that it should have a large surface-to-volume ratio, which would provide enough accessible reaction site for the DA and NE oxidation, and a high carrier mobility, which could enhance the charge transfer between the DA/NE molecules and the catalyst surface to accelerate the oxidation reaction. 2D materials possess favorable dimensions and large surface-to-volume ratios that enhance the performance of biosensors. Most reported 2D materials are n-type, which could be attributed to the strong electron doping effect of interfacial charge impurities and intrinsic defects. On the contrary, P-type 2D semiconductors with desired properties are still limited in both the amount and studies because the p-type conduction of 2D materials is always haunted by the fermi level pinning at the metal/2D materials interface, prohibiting the injection of holes. There are only a few intrinsic p-type 2D semiconductors such as WSe₂, black phosphorus (BP), tellurene, and MnS₂ etc., where many of them suffer from ambient condition stability due to the unstable dopants, vulnerable 2D interfaces, and environmental impacts, which hinders their applications for human wearables. Interestingly, a Te nanowires coated GCE was fabricated to detect DA in PC12 cells. They discovered the partial donation of the lone pair electrons from the nitrogen atoms in DA to the Te atoms facilitates the efficient adsorption of DA so that an increased sensitivity and selectivity of DA could be observed in the presence of AA and UA. However, due to the limited exposure of Te atoms on the surface of Te NWs, the highest detectable concentration of DA was only 1 μM in their report, which is irrelevant to the DA concentration in body fluidics. Moreover, known systems do not demonstrate the sensing of DA from real human sweat.

Accordingly, there is a continuing need for an electrochemical sensor system that enables enhanced absorption and desorption of DA molecules compared to known sensors.

### SUMMARY

In concordance with the present disclosure, an electrochemical sensor system that enables enhanced absorption and desorption of DA molecules compared to known sensors, has surprisingly been discovered.

An electrochemical sensor system includes a substrate, a working electrode, a counter electrode, a reference electrode, and an adhesive layer. The working electrode includes a tellurene-based material. The working electrode may also include metallic nanoparticles disposed on the tellurene-based material. The counter electrode may include a carbon-based material. The reference electrode may include a silver/silver chloride-based material. The adhesive layer may be permeable to sweat. The system may further include a polydimethylsiloxane (PDMS) layer that includes microfluidic channels. The working electrode, the counter electrode, the reference electrode, and the PDMS layer may be sandwiched between the substrate and the adhesive layer. The electrochemical sensor system may be configured to detect a neurotransmitter from sweat.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a top perspective view of a wearable tellurene-based biosensor patterned on a flexible PET substrate, according to one embodiment of the present disclosure;

FIG. 2 is a top plan view of the sensor system having a working electrode that includes tellurene and a gold material, a reference electrode that includes a silver/silver chloride material, a counter electrode that includes a carbon-based material, according to one embodiment of the present disclosure;

FIG. 3 is an expanded front elevational view of the sensor system illustrating a substrate, a working electrode, a reference electrode, a counter electrode, a PDMS layer, and an adhesive layer being disposed on the epidermis layer of a user, according to one embodiment of the present disclosure;

FIG. 4 is expanded front elevational view of drop-casting a Te-Au/GCE biosensor, further depicting a bare GCE layer, a Te/GCE layer, and an Au@Te/GCE layer, where the disk (central circular region) diameter includes a glassy carbon active area surrounded by a polytetrafluoroethylene (PTFE) rod, according to one embodiment of the present disclosure; and

FIG. 5 is a flowchart depicting a method of manufacturing the sensor system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the FIG. is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

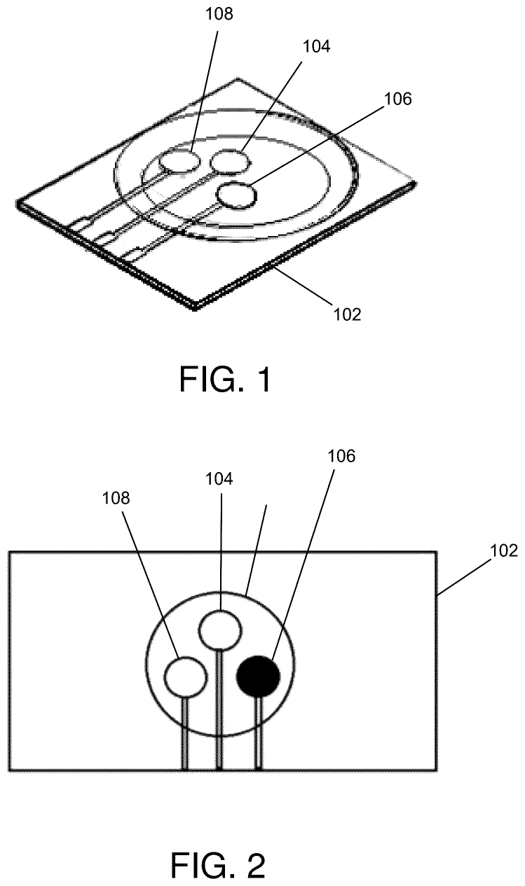

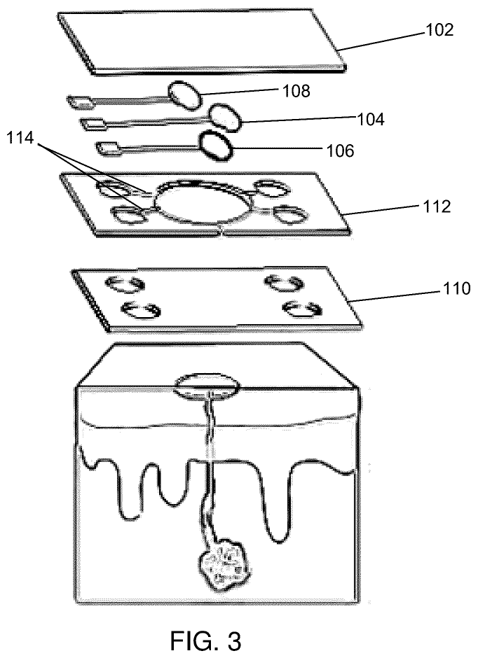

As shown in FIGS. 1-3, an electrochemical sensor system 100 includes a substrate 102, a working electrode 104, a counter electrode 106, a reference electrode 108, and an adhesive layer 110. The working electrode 104 includes a tellurene-based material. The working electrode 104 may also include metallic nanoparticles disposed on the tellurene-based material. The counter electrode 106 may include a carbon-based material. The reference electrode 108 may include a silver/silver chloride-based material. The adhesive layer 110 may be permeable to sweat. The system 100 may further include a polydimethylsiloxane (PDMS) layer 112 that includes microfluidic channels 114. The working electrode 104, the counter electrode 106, the reference electrode 108, and the PDMS layer 112 may be sandwiched between the substrate 102 and the adhesive layer 110. The electrochemical sensor system 100 may be configured to detect a neurotransmitter from sweat. One skilled in the art may select other suitable ways to provide the electrochemical sensor system 100, within the scope of the present disclosure.

Tellurene, a 2D p-type semiconductor was chosen to form part of a sensor's electrodes because 2D tellurene has a higher ratio of exposed Te atoms than other nanostructures such as nanowires or nanotubes. The interaction between the Te and the nitrogen atoms would allow the easy absorption and desorption of the DA molecules to tellurene. Moreover, tellurene has high hole mobility. To better boost the performance of the electrochemical sensor system 100, further decoration with metal nanoparticles like gold nanoparticles may be implemented as this may increase the conductivity of the sensor. In a specific example, Au nanoparticles/Tellurene, such as a 0D/2D mixed dimension hybrid structure, may be provided and manufactured through a rapid in-situ transformation process, and further integrated into a biosensor to enable DA and NE detection.

Provided as a non-limiting example, the electrochemical sensor system 100 may exhibit a sensitivity of around 1.32 μA/μM and around 33.2 μA/μM for the selective and sensitive detection of DA and NE, which are remarkably superior to previous works, which were all tested in artificial sweat, blood, saliva and serum. Through in situ functionalization and surface engineering with Au nanoparticles, the electrochemical sensor system 100 exhibits a good linearity of oxidation current against the concentration of DA over a wide range of 1-2.8 mM in PBS, which presents the largest known range reported. The large detection range demonstrates the excellent antifouling ability of the electrochemical sensor system 100, which may be extremely important for NTs detection in complex biofluids because nonspecific adsorption or fouling at the electrode interface via the innumerable biomolecules present in various sample types (i.e., serum, urine, blood/plasma, and saliva) can drastically obstruct electrochemical performance, increasing background “noise” and diminishing both the electrochemical signal magnitude and specificity of the biosensor. Due to the specific binding between Te and nitrogen atoms on biomolecules (DA/NE), the electrochemical sensor system 100 shows an enhanced selectivity of DA in the presence of uric acid and ascorbic acid with large concentrations (1 mM, larger than the ~20-60 μM range in human body fluids). Besides, the electrochemical sensor system 100 also exhibits the ability to detect simultaneously L-dopa, DA, and NE, which may be a great platform to be readily adaptable for studying the dynamics of various signaling physiological and psychological signals throughout the body, including dynamics of medical doses of AD or Parkinson's patients, persons with psychosocial disabilities, health groups with mental health concerns or vertebrate and mollusk, and possibly even in plants. Most significantly, it was demonstrated the continuous in vivo monitoring of DA and NE concentrations in real human sweat using wearable sensors for the first time.

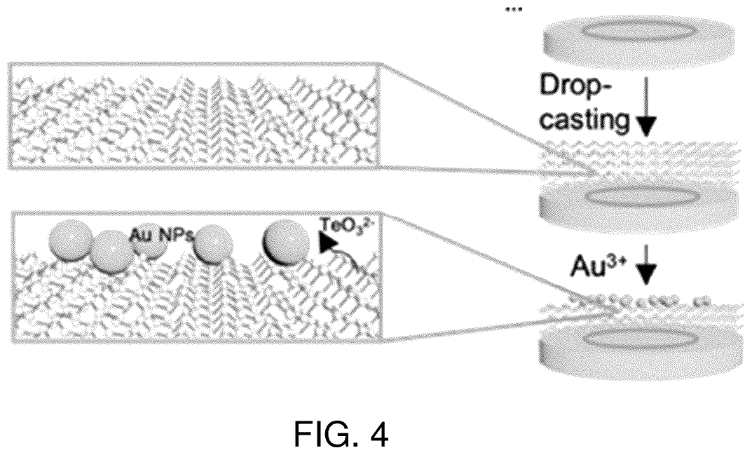

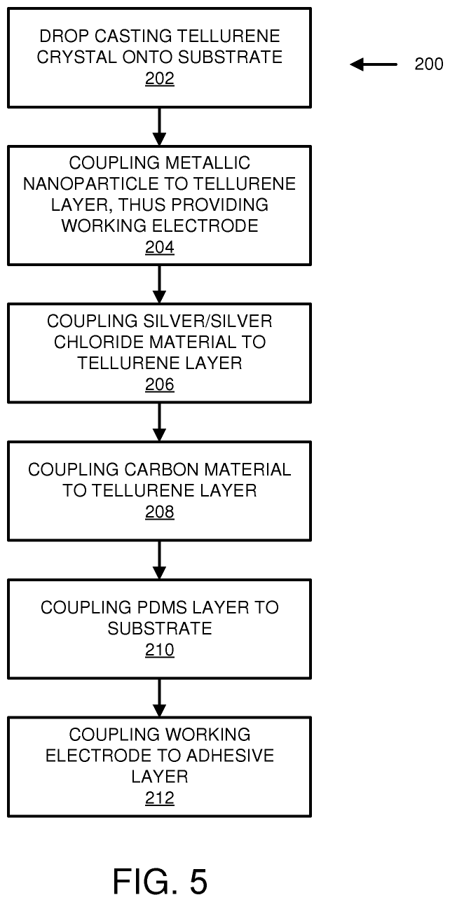

The electrochemical sensor system 100 may be manufactured in various ways. Provided as a non-limiting example, one process for preparing the electrochemical sensor system 100 is shown in FIGS. 4-5. The method 200 may include drop casting a tellurene crystal on a substrate 102, thus forming a tellurene layer. Next, the method 200 may include coupling a metallic nanoparticle(s) to the tellurene layer, thus providing a working electrode 104. The metallic nanoparticle(s) may include a gold material. Afterwards, the method 200 may include coupling the working electrode 104 to an adhesive layer 110. In certain circumstances, the method 200 may also include coupling a silver/silver chloride-based material to the tellurene layer, thus providing a reference electrode. The method 200 may also include coupling a carbon-based material to the tellurene layer, thus providing a counter electrode. In a specific example, the method 200 may also include coupling a polydimethylsiloxane (PDMS) layer 112 to the substrate 102 before the step of coupling the working electrode 104 to the adhesive layer 110.

In one specific example, tellurene crystals were synthesized through an aqueous reaction following our reported procedures and transferred to modify the GCE surface through drop-casting and air-dry at room temperature. Solutions with controlled concentrations of chloroauric acid (HAuCl₄) were subsequently introduced to derive gold nanoparticles on the tellurene layer. The color transition from silver gray to yellow in tellurene flakes before and after introducing chloroauric acid also indicates the decoration of tellurene with gold nanoparticles. A skilled artisan may select other suitable ways for preparing the electrochemical sensor system 100, within the scope of the present disclosure.

Group VI tellurium (Te) has a unique chiral chain lattice where weak bonds stack individual helical chains of Te atoms and spiral around axes parallel to the [0001] direction. Each tellurium atom is covalently bonded with its two nearest neighbors on the same chain. The obtained tellurene flakes have edge lengths ranging from 50 to 100 μm and thicknesses ~60 nm. High-resolution transmission electron microscopy (HRTEM) characterization showed that the tellurene flakes laterally grow along [0001] and [12̅10] directions, with vertical stacking along [1010] direction, which is consistent with previous reports. TEM and Energy-dispersive X-ray spectroscopy (EDX) characterizations confirm that gold nanoparticles with controlled dimension (diameter ~10 nm) and spatial densities (coverage range 74%-93%) were homogeneously loaded on the (101̅0) surface of tellurene. Elemental mapping images of Au and Te by high-angle annular dark-field STEM energy-dispersive X-ray (HAADF-STEM-EDX) spectroscopy revealed that the 20-nm layer gold nanoparticles were uniformly dispersed on the surface of tellurene flakes. XRD measurements also confirm the phase and crystallographic structure of tellurene and gold nanoparticles. The coverage of gold nanoparticles on the tellurene surface can be modulated by controlling the precursor concentration, tellurene width, and reaction time. The number of gold atoms deposited on tellurene depends on the surface Te atoms available. Therefore, the in-situ reduction of gold nanoparticles is self-limited by consuming surface Te atoms. Such a self-limiting mechanism mitigates the aggregation and non-uniform size of nanoparticles encountered in previous reports and allows a controlled partial exposure of the tellurene (101̅0) surface, which is critical for successfully detecting targeted neurotransmitters. The energy band diagram and density-functional-theory (DFT) calculation indicate the formation of ohmic contact between gold nanoparticles and tellurenE. Bader charge analysis indicates that the resultant flow of electrons from tellurene to gold induces hole accumulation and improves the conductivity in p-type tellurene.

In certain circumstances, the electrochemical sensor system 100 includes an Au/Te hybrid nanostructure that is approximately 1 μm thick. During the reduction process, the Au³⁺ solution infiltrates the interlayer of Te flakes, leading to the vertical linkage of the flakes and a consequent increase in the conductivity of the tellurene film. In order to substantiate the hypothesis regarding the presence of Au NPs on the surface, EDX analysis was employed. However, an increase in the thickness of Te flakes was observed in comparison to pure Te. This behavior can be attributed to the stacking of 2 or 3 flakes. Consequently, while the diffusion of Au³⁺ solution may occur within the gaps between the flakes, it is impeded in densely packed flakes.

The anisotropic electron transfer path of Au@Te heterostructures interlocked framework in the edge and basal planes were observed. Reactant redox probes, after electro-oxidation, donate electrons, and the electrons will be transferred to nearby nanoparticles through surface conductive pathway rather than bulk in the basal plane. In the edge plane, the electrons will follow the Au NP conductive pathway to finish the charge transfer through multiple paths established by porous 2D materials and metal interlocked framework structure. Finally, these charges will be collected by the current collector (GCE). Charge transfer resistance (Rct) at the electrode is a vital parameter to assess the charge transfer rate of the electrochemical biosensor, which can be investigated by electrochemical impedance spectroscopy (EIS) measurements though three-electrode system. The semicircle diameter of EIS is equal to Rct in the Nyquist diagram. The bare GCE has a large semicircle in the high-frequency section. Then, following the loading of the Te on the GCE, Rct increased compared to the bare GCE. This can be attributed to the metalloid characteristics of tellurene. When Au nanoparticles loaded with a coverage rate of 74%, the semicircle decreased markedly (over 90%) because the Au nanoparticles improved charge transfer. This change in resistance indicates that gold reduction was an efficient method to improve the charge transfer rate of semiconductor Te property.

The cyclic voltammograms (CV) studies were carried out to investigate the electrochemical behavior of the bare GCE, Te/GCE, and Au@Te/GCE recorded in the presence of 1 mM ferro/ferricyanide Fe (CN)₆^{3-/4-} redox probes, one of the most extensively studied redox couples in electrochemistry, in 0.1 M KCl. Modification of the Au@Te onto the GCE surface increases the redox peak current of [Fe (CN)₆]^{3-/4-} compared with unmodified GCE. This enhanced electrocatalytic activity could be attributed to the higher surface area of the 2D nanostructured hetero-compounds with excellent electrical conductivity and catalytic ability, which could facilitate the [Fe (CN)₆]^{3-/4-} redox reaction. To further investigate the possible electrode reaction mechanism, the study of scan rates' effect on peak current has been done using the CV method. A progressive increase in both anodic and cathodic peaks were observed with the increase of scan rate, indicating that electrode reaction is reversible. In addition, there is also an excellent linear relationship between the cathodic peak currents and the square root of scan rate in the range of 10-150 mV/s. It reveals that the reactions on the Te@Au/GCE were controlled by diffusion process. The active surface areas of Te@Au/GCE can be estimated using the Randles-Sevcik equation. The active surface area of Te@Au/GCE was determined to be 0.3885 cm², which was higher than other reports. 2D tellurene possesses favorable size characteristics and a large surface-to-volume ratio that increases the electrolyte accessible surface area and the utilization of tellurene active sites, leading to a much-improved electrochemical activity. There is not much decrease after 5000 tests, indicating the modified electrode's decent electrochemical stability. It is attributed to the excellent reversibility of the reaction on the electrode and metalloid characteristics of tellurene and well-dispersed Au NPs. This also proves that the interface of the electrochemical sensor system 100 may provide fast electron exchange without participating in the redox reaction, and the electrochemical equilibrium can be quickly established, facilitating the long-term use and fast response of the biosensor.

The electrochemical behaviors of bare GCE, Te/GCE, and Au@Te/GCE electrodes in PBS buffer in the presence of DA were recorded. In this process, DA was oxidized to dopamine quinone (DAQ) by exchanging 2 electrons and 2 protons under positive overpotential with various electrodes and produce faradaic current. The voltammograms of DA on Au@Te/GCE show an intense peak current at a lower potential (0.285 V) compared to the pair of redox peaks from GCE (0.376 V). This pair of reversible redox peaks shows that a lower overpotential was required for the DA electro-oxidation process. Interestingly, Te/GCE has a decent electrocatalytic activity of DA, even considering its low intrinsic carrier concentration. According to the Butler-Volmer equation, the overpotential of Te/GCE for DA electro-oxidation decreased compared to bare GCE, indicating the activation energy decreased, which suggests tellurene has a specific interaction with DA molecules and lowers the reaction energy barrier. Te atoms of tellurene interact with nitrogen atoms on DA, which will help improve the sensitivity. The lone pair of electrons on nitrogen atoms will form a coordination tellurium-nitrogen π-bonding bond with the Te atoms, facilitating the efficient adsorption of DA on the surface of Te@Au/GCE. In addition, the TeO₃²⁻ residuals after the in-situ reaction of gold nanoparticles can give a repulsive force to negatively charged UA and AA molecules, which is another reason for enhanced selectivity to DA of Au@Te/GCE electrode.

In order to evaluate the selectivity of the electrochemical sensor system 100, differential pulse voltammograms (DPV) of DA were carried out in the presence of AA and UA in the PBS (pH=7.0) solution using GCE, Te/GCE, and Au@Te/GCE. Due to the large concentration (20-60 μM) of AA and UA in sweat and the similar oxidative peaks (typically on 0.4-0.6 V) for DA, AA, and UA, the selectivity of detecting DA from real sweat is challenging for bare GCE105 and other sensor electrodes in previous efforts. Here, a relatively high concentration (1 mM DA, AA, and UA, respectively) of analytes was chosen to compare the responses of 3 kinds of electrodes (GCE, Te/GCE, and Au@Te/GCE). It was observed that bare GCE has poor DA selectivity because the current peaks of DA and AA overlap in the potential range of 0.4-0.6 V. When the Te/GCE electrode was applied, the current of DA was much higher than GCE, but the three peaks also cannot be distinctly defined due to the near positions of each one. As discussed before, Te atoms of the tellurene surface tend to interact with nitrogen atoms on biomolecules, which will help to improve the electrode sensitivity. However, pure Te might not satisfy the charge transfer rate. Regarding Au@Te/GCE, the AA, DA, and UA peaks were separated at 0.205V, 0.39V and 0.57V, respectively. Due to Au nanostructures loaded on tellurene, the conductivity and electrocatalysis of the electrochemical sensor system 100 may be improved significantly, leading to a high charge transfer process and distinct current peaks of DA, AA, and UA.

Furthermore, we explored the linear detection range of the electrochemical sensor system 100 obtained by the DPV measurements using the Au@Te/GCE electrode in pH=7.0 PBS upon successive addition of different concentrations of DA solutions. The anodic current of DA in the presence of AA and UA are increased linearly with the concentrations of DA in the range of 1-500 μM with a correlation coefficient of 0.9922 and in the range of 500-2875 μM with a correlation coefficient of 0.9910. The DA, UA, and AA concentrations we used in the experiments are 1 μM, 1 mM and 1 mM, respectively. It suggests that the electrochemical sensor system 100 may still detect DA even in 1000 times higher AA and UA than DA. As the DA concentration (cDA) increases, the surface of Te is saturated with DA. The increase in the peak current becomes slower, thus resulting in another regression equation. Conversely, as cDA increases, the DPV peak shifts to more positive values. These shifts are generally attributed to a decreased diffusion coefficient and slower electron transfer when cDA increases 141. Thus, a more positive potential is needed to overcome the kinetic barrier. In the reported physiological-relevant range of DA (0.1-100 μM) 97, our biosensor shows negligible shift (5 mV). All electro-active metabolites with similar chemical structures are excluded from this range. This is mainly attributed to the high accumulation of holes in Au@Te/GCE, lowering the DA oxidation energy barrier. If the semiconductor Fermi level is lower than the redox Fermi level (EF, sc<EF, redox), as is typical for p-type semiconductors here, it is the holes that flow from the semiconductor to the electrolyte, thereby raising the semiconductor Fermi-level until it equals the redox Fermi level142. At equilibrium, the interface of tellurene is considered fully “inert” without the charge transfer process and is thus capable of accumulating an extremely high surface charge (holes) concentration, making the p-type semiconductor highly conductive or even superconductive. At a positive overpotential, these highly accumulated holes among p-type tellurene will be “active” due to the self-gating effect (negative charge gating), resulting in anodic reaction (DA electro-oxidation). Therefore, at positive overpotential, the anodic current density will be greater than at equilibrium at relatively low overpotential using the electrochemical sensor system 100 and thus achieve excellent sensitivity.

The energetics have been computed using DFT to optimize the sensing performance and understand the interaction with DA in the Au@Te/GCE electrode. We studied the binding energy of DA using DFT calculations on tellurene and Au (111) surfaces. The DA molecule interacts with the surface through both N and OH bonds. However, the dangling OH bonds in the real electrochemical system will form H-bonds with the surrounding water. Thus, nitrogen atoms dominate the effect when DA molecules interact with electrodes. Using Bader charge analysis, we observe that the nitrogen atom donates electrons to the tellurene and Au surfaces. The binding energy of DA to Au is too strong (1.24 eV) compared to the binding energy between Te and DA (0.81 eV), so the electrode is easily deactivated because the oxidated products (quinone) would be difficult to desorb from the electrode surface. Te atom can form a weak π bonding with the N atom of DA, which is ideal for operations of the electrochemical sensor system 100 because the new DA molecules can access the surface of the electrochemical sensor system 100 due to the easy desorption. Therefore, to obtain the best biosensor performance, it is necessary to balance the higher conductivity provided by gold particles and the weak binding of exposed Te atoms to DA molecules. Au NPs should partially cover the Te surface to play the synergistic effect. Compared to the previous approaches of synthesizing metal nanoparticles in the organic phase at high temperature, the rapid in-situ reduction in our work provides a more favorable route at ambient temperature in the aqueous phase without any surfactants. Problems of the surfactant's method are the resulting solutions' low gold nanoparticle content (<0.01 M) and the restriction to water as a solvent. Because the charge-stabilized particles quickly undergo irreversible aggregation upon the addition of electrolytes and nonpolar surfactants, particles are difficult to be surface-derivatized further and also put the barrier to be electrochemical electrode in electrolytes. In addition, without surfactants, the coverage of gold nanoparticles can also be controlled by the process to enhance the performance of the electrochemical sensor system 100 further.

Three aspects affect the final coverage of Au NPs on the tellurene surface: chloroauric acid concentration, reaction time, and width of the tellurene flake. When the width of the tellurene flake is fixed, more significant chloroauric acid concentration and longer reaction time will lead to a higher coverage of Au NPs. When the width of the tellurene flake is constant, different concentrations of chloroauric acid for reduction and different reaction time were adopted to synthesize the electrochemical sensor system 100. The anodic peak (ipc) current of DA was recorded in PBS. The ipc increased along with the increased Au3+ concentration and reaction time and reached to “apex” when Au3+ concentration was 4 mg/mL and reaction time was 60 s (coverage rate: 86.16%). When these two parameters were increased, the ipc decreased, and the Au NPs coverage increased to 93.07%. This decrease may be attributed to the reduction and/or elimination of DA molecules and tellurene surfaces due to the large coverage of Au NPs. When the Au3+ concentration was fixed at 4 mg/mL, tuning the Te flake width and reaction time can modulate the coverage rate of Au NPs. When the width of tellurene increases, a longer reaction time would be needed to achieve the best coverage rate (86.16%), so the “apex” appears in the top right corner. Compared to the previous works using nanomaterials-modified glassy carbon electrodes for detecting DA in PBS, the Au@Te/GCE biosensor can achieve a comparable limit of detection (2 nM), sensitivity (0.083 μA/μM), wider linear response range (1 μM-2.875 mM), demonstrating the superior performance of the hybrid electrode fabricated by the in-situ reduction process. Mn—MoS243 and Ag-rGO array were applied as working electrodes 104 to detect DA in artificial sweat/serum, but the feasibility of those sensors in detecting DA/NE from real sweat is unclear. This biosensor also exhibits ultralow LOD of NE detection (1 nM), good linear response (1 nM-400 nM), and the highest sensitivity (33.2 μA/μM) compared to state-of-the-art NE biosensors. The electrochemical sensor system 100 shows good electrochemical stability, good response behavior (recovery rate=3.7/2.8, and repeatability (relative standard deviations R. S. D.<3.0 %) in artificial plasma mainly due to the excellent absorption-desorption process.

There are no known reports on electrochemical sensing DA and NE in real human sweat. As a proof of concept and to demonstrate the compatibility of the electrochemical sensor system 100 for integration with wearable platforms, an all-integrated sensor on a flexible polyester (PET) substrate 102 was fabricated. Traditional electrochemical setups, with bulky reference electrodes 108 (REs), can be cumbersome and pose a challenge in the pursuit of miniaturization and portability. Hence, these setups are not conducive to point-of-care monitoring. Here, the electrochemical sensor system 100 may use a tellurene-based material as the working electrode 104 (WE), Ag/AgCl as the reference electrode 108 (RE), and carbon paste as the counter electrode 106 (CE). The sweat monitoring system 100 based on Au@Te (SMS-Au@Te) may contain three layers, including a sensor on a polyester substrate 102, microfluidic channels 114 patterned on a polydimethylsiloxane (PDMS) layer 112, and an adhesive layer 110 with sweat input pattern. As sweat flows into the system 100, the electrochemical sensor system 100 continuously measures sweat's DA and NE concentration dynamics.

The amperometric i-t curve technique was adopted for real-time analyte monitoring. The respective current responses of DA were obtained (sensitivity: 1.32 μA/μM and LOD: 0.0105 nM), NE (sensitivity: 33.2 μA/μM and LOD: 0.0463 pM), measured amperometrically within their corresponding physiological relevant range [DA:0 to 20 μM, NE:0 to 1 μM performed in phosphate-buffered saline (PBS)]. The LOD is calculated based on S/N=3:1. The electrochemical sensor system 100 may also detect L-dopa. According to the Cottrell equation, the electrochemical sensor system 100 responses are linear to the corresponding analyte's concentration.

Differential pulse voltammetry (DPV) is chosen to evaluate the ultralow level of sweat DA and NE based on the amplitude of the oxidation current peak. It can simultaneously monitor these two NTs using only one working electrode 104. Two distinct current peaks at ~0.05 V and ~0.39 V from the DPV scans correspond to the oxidation reactions of NE and DA, respectively in real sweat samples. The sweat sensor was evaluated using human subjects in compliance with all the ethical regulations under a protocol (IRB-2022-802) (See methods in detail). No other distinct peaks show that the Tellurene-based DA and NE sensor of the present disclosure has excellent selectivity over other analytes in sweat at physiologically relevant concentrations. The electrochemical sensor system 100 displays superior electrochemical performance over screen-printed carbon and silver electrodes due to the low selectivity and biofouling of these electrodes47,139. To verify the concentrations of DA and NE in human sweat samples, high-performance liquid chromatography mass spectrometry (HPLC-MS) analysis was adopted to analyze the sweat sample quantitively. LC-MS characterizes biomolecules according to retention time (RT), the accurate mass of the molecular ion derived from the full mass spectrum (MS1), and the fragmentation pattern determined by tandem mass spectrometry (MS2)154. The RT for DA and NE are 6.516 min and 6.901, consistent with the standard internal dopamine-d 4 and norepinephrine-d6, respectively. Multiple reaction monitoring (MRM) mass spectroscopy, a highly sensitive quantitative protein detection technology, was adopted. The transition from m/z 158.3 to m/z 141.2 is used to obtain the extracted ion chromatogram (XIC) for dopamine and m/z 176.3 to m/z 158.2 for norepinephrine. The calculated concentrations of DA and NE are 1.59 μM and 0.468 μM. This is first time to report the physiological concentrations of DA and NE in human sweat and provide the foundation for wearable sweat sensor of neurotransmitters in the future works.

To further validate the concentration range of DA and NE in human sweat, 13 sweat samples from different human subjects after exercise were tested using our fabricated electrochemical sensor system 100. The concentration of DA ranges from 0.05-8.64 μM and 11.00-102.90 nM for NE, which is close to urinary samples (0.3-3 μM of DA and 90-500 nM of NE)97. This concentration is larger than in human plasma (~0.08 nM of DA and ~1.5 nM)98 and saliva (~0.17 nM of NE and ~0.45 nM of DA)99. This may be because the sweat we detected was obtained after the person exercised. Many relevant studies have demonstrated that DA and NE concentrations increase significantly or even more than 10-fold in biofluids such as plasma and saliva after physical exercise. The accuracy of the wearable sensor 100 for analyzing real sweat samples using high-performance liquid chromatography (HPLC) was validated. We obtained a high correlation for DA and NE sensing for 13 subjects, suggesting the reliability of the sensor measurements. However, there are some differences due to HPLC lag time and the complex pre-processing procedure of sweat samples.

Monitoring stress levels is important for various groups, including those with demanding jobs (such as emergency workers, police officers, firefighters, and members of the armed forces), athletes, and people with specific medical conditions. Physiological and psychological consequences due to exposure to acute or chronic stress can be vast and detrimental, manifesting in various physical and chemical ways. Some of the harmful effects due to prolonged periods of stress are depression, neurological disorder or breakdown, cardiothoracic disease, weight variations (loss or gain), and hyperglycaemia.

The fully integrated system 100 could be worn on the human body to achieve real-time and longitudinal monitoring of the DA/NE levels during various daily activities. Real-time measurement of sweat DA and NE from the arm of a healthy individual in a cycling exercise was demonstrated. An increased trend in sweat DA and NE concentrations was observed, likely because the sympathetic nervous system plays a crucial role in regulating arterial blood pressure and blood flow during exercise. The sympathetic stimulation induces the release of NE from the adrenal medulla to promote sweat secretion from both eccrine and apocrine glands.

We speculate that NTs in sweat originate from the diffusion of the transmitter (DA/NE) secreted by the sympathetic nerve system regulating the sweat gland, similar to salivary catecholamines (CA). Findings for abnormal values (more than ten times higher than normal) and the elevation of salivary CA to this level in subjects reporting strong anxiety or intense nervousness suggested that the system 100 is useful for the measurement of human stress. Monitoring the subjects'metabolite profiles (DA and NE) through various daily events and in different settings, including mental stress loading and after drinking coffee were completed. For the mental stress stimuli, the Stroop color and word test (SCWT) was chosen as a neuropsychological test extensively used to assess the ability to inhibit cognitive interference. The concentration of NE in the sweat increased significantly (over 17-fold) relative to baseline at 35 min and started to decrease after 35 min. In contrast, the concentration of DA did not change significantly throughout the process. This monitoring indicates mental stress increased sweat NE, which points to an increase in sympathetic nervous system activity. However, the mental effort does not significantly impact the DA excretion since a significant portion of DA in plasma is conjugated as sulfates and glucuronides. The sweat excretion of free DA may reflect the changes in the total pool of plasma DA.

In another test, coffee was consumed, and then the NE/DA levels were recorded after 10 mins. Dopamine increases 15 min after taking coffee and fluctuates on the high concentration (about 2-folds compared to initial 15 min testing results) between 15-35 mins, and finally starts declining after 35 mins. Some reports claim that consuming caffeine capsules will increase DA, and that caffeine needs at least 4-6 hours in the body to be completely metabolized. The DA level in sweat during the whole test fluctuated at a high concentration compared to the level during SCWT.

In another scenario, a custom-made adjustable wood platform was built to mimic the rooftop on which the simulated shingle installation was conducted. A lift with adjustable forks would raise one side of the roof simulator. The experiment was performed in the heating, ventilation, and air conditioning (HVAC) and indoor air quality lab. A complete dynamic trial consisted of 7 phases—reaching for shingles, placing shingles, grabbing the nail gun, moving to the first nailing position, installing shingles by nailing, replacing the nail gun, and returning to the upright position. During the whole process, the participants wore sweat-sensor-integrated helmets and recorded the DA/NE concentrations through a Bluetooth module. The DA firstly increased due to the physical stress loading and then decreased. Before stopping the heat and roofing work, the DA increases again and continues to increase after work due to the pleasurable stimuli and reinforces behaviors that lead to pleasurable outcomes. NE was relatively high at the beginning and then dropped slightly. However, it was always maintained at a very low concentration (1-4 nM), indicating that the person was not exposed to high mental stress levels.

Overall, DA/NE levels in human sweat are a valuable index for assessing human physiological and psychological situations. Potential applications for these findings include training in sports where biomarkers are required to evaluate and assess the stress that sports training might cause that may result in injury. Athletes' health and well-being are impacted by intense and ongoing physical training in sports due to the psychological and physiological stress it causes. To use sweat as a possible biological fluid for stress biomarker assessment, a non-invasive sampling technique is developed in this work. Furthermore, stress can impact a sportsperson's health and performance in football and other sports. Heart attack and kidney failure have also been described as stress-related consequences, in addition to muscle damage that can result from the immune system's reaction to stress.

2D tellurene was synthesized utilizing analytical-grade Na₂TeO₃ (0.00045 mol) and an amount of Polyvinylpyrrolidone (PVP) were placed into deionized water (33 ml) at room temperature under magnetic stirring to form a homogeneous solution. The well-stirred solution was poured into a Teflon-lined stainless-steel autoclave, which was then filled with an aqueous ammonia solution (25%) and hydrazine hydrate (80%). The autoclave was sealed and maintained at 180 °C for 30 h. The autoclave was then cooled to room temperature naturally. The resulting solid silver-grey products were precipitated by centrifugation at 5,000 rpm for 5 min and washed three times with distilled water and acetone (v/v=3:1) (to remove any ions remaining in the final product). For the thinning process using an alkaline growth solution, as synthesized 2D tellurene solution (1 ml) was mixed with acetone (3 ml) at room temperature. thin 2D tellurene was obtained by centrifugation at 5,000 r.p.m. for 5 min. Finally, disperse the products in 1 mL Isopropyl Alcohol/distilled water (v/v=3:1).

The glassy carbon electrode (GCE) surface was polished to mirror-like with 0.3 and 0.05 μm Al2O3 powder, and then successively sonicated in water, ethanol, and ethanol each for 3 mins. After the GCE was dried with N 2 flow, 5 μL 2D Te as-prepared dispersion solution was dropped on the pretreated GCE surface and dried at room temperature. Then 10 0-7 mg/mL HAuCl4/DI water solution was added on the surface of 2D Te modified GCE. After 5 s, The Te@Au/GCE was gently washed with doubly distilled water and then dried at room temperature.

All electrochemical experiments were conducted with a CHI660C workstation (Shanghai Chenhua, Shanghai) in a three-electrode system, using a coiled platinum wire as the auxiliary electrode, a saturated calomel electrode (SCE) as the reference electrode 108, and the as-fabricated electrode as the working electrode 104. CV was recorded in the presence of 1 mM [Fe (CN)6]3−/4− in 0.1 M KCl at a scan rate of 100 mV/s for 5000 cycles. Their selectivity toward DA over AA and UA was verified by conducting CV over the potential range from −0.2 to +0.6 V at a scan rate of 100 mV s−1 in PBS. The interference test and the detection of DA in the presence of 1 mM AA and 1 mM UA obtained by differential pulse voltammetry (DPV) was conducted over the potential range from −0.2 to +0.6 V at a scan rate of 100 mV s−1 and a pulse amplitude of 50 mV in PBS. Impedance measurements were performed with the frequencies swept from 0.01 to 100000 Hz. A Randle's equivalent circuit was used to fit the obtained impedance spectra. To make sure that the added DA was fully mixed, the electrolyte was stirred for about 1 min. CV was carried out until the peak current was stable and then the DPV was performed immediately.

Periodic Density Functional Theory (DFT) calculations were performed using the Vienna Ab Initio Simulation Package (VASP)169,170. The core electrons were modelled using the Projector Augmented Wave (PAW) potentials171. The energy cutoff for the planewave basis was set to 500 eV and the Monkhorst-Pack k point scheme was used for all the calculations. The (111) surface of Au and the (1010) surface of Te were modelled from their corresponding converged bulk structures172. The Au/Te interface was made such that interfacial strain in non-pseudomorphic, periodic overlayer structures is minimized. The generalized gradient approximation (GGA) was employed in the form of the Perdew-Burke-Ernzerhof (PBE) functional173. The charge transfer was quantified using the Bader charge analysis described by Henkelman and coworkers174. The van der Waals interactions were corrected using the DFT-D3 method175,176.

In certain circumstances, the wearable sweat sensor may be fabricated on a flexible PET substrate 102. For instance, the following includes a non-limiting example of manufacturing the electrochemical sensor system 100. Au@Te was used as the WE, carbon conductive paste, and silver/silver chloride (60/40) paste (Sigma-Aldrich) as CE and RE, respectively, through screen-printing. All electrodes were cured at 100 °C in an oven for 30 mins. The conductive circuit was printed by the Voltera V-One printer. A 3D printer printed the microfluidic layer. Moreover, the adhesive layer 110 that came into contact with the skin was fabricated by cutting the medical tape. The Sensit BT (PalmSens BV) was used to carry out the DPV and i-t measurement and transferred the acquired data through Bluetooth.

A non-limiting example of the sweat sensor was evaluated using human subjects in compliance with all the ethical regulations under a protocol (IRB-2022-802) approved by the Institutional Review Board (IRB) at Purdue University. The participating subjects (age 18-65) were recruited from the Purdue University campus. All subjects gave written informed consent before participation in the study. A stationary exercise bike was used for cycling trials. The subjects are asked to cycle at 60 rpm for 40 min. Every 10 minutes, the differential pulse voltammetry (DPV) scan initiates a transient voltage bias between the reference and working electrodes 104 by using Sensit BT. The acquired current-voltage data will be transmitted to the computer remotely and converted to the concentration levels of analytes using Origin. Meanwhile, sweat samples are collected periodically from the forehead or neck of the subjects using centrifuge tubes and then centrifuged at 6,000 rpm for 15 min. The sweat samples are frozen at −20 °C for further testing and validation via High-performance liquid chromatography (HPLC) analysis.

The 100 μL sweat sample was dissolved in the 100 μL acetonitrile (ACN) with 50 μL 100 μg/mL internal standard of dopamine- 4 hydrochloride solution and Norepinephrine-D6 hydrochloride solution (Sigma-Aldrich). The mixture was centrifugated under 3000 rpm, and then the supernatant was transferred into HPLC vials equipped with a 200 μL V-shape glass insert (both Macherey-Nagel GmbH & Co. KG) and analyzed by LC-MS/MS.

A stroop color and word test (SCWT) was used to induce stress. This test consists of two sessions: congruent (non-conflict task) and incongruent (conflict task). In the congruent session, the color of the box and the word written in the box are the same. In the incongruent session, the color of the box and the words written in the box are different. The test lasts 45 min under the testing temperature of 40 °C to induce human sweat during the process. The data was obtained every 5 mins to evaluate the DA and NE levels in sweat. The first 25 minutes were used to sweat induction and test the baseline (pre-test), and the SCWT test lasted 10 minutes (2 mins for congruent session and 8 mins for incongruent session), starting at 25 minutes and stopping at 35 minutes.

A custom-made adjustable wood platform was built to mimic the rooftop on which the simulated shingle installation was conducted. A lift with adjustable forks would raise one side of the roof simulator. The experiment was performed in the heating, ventilation, and air conditioning (HVAC) and indoor air quality lab. A complete dynamic trial consisted of 7 phases—reaching for shingles, placing shingles, grabbing the nail gun, moving to the first nailing position, installing shingles by nailing, replacing the nail gun, and returning to the upright position. The participants wore sweat-sensor-integrated helmets during the process and recorded the DA/NE concentrations through a Bluetooth module.

The drawings described herein are for illustrative purposes only of selected embodiments and not all possible implementations and are not intended to limit the scope of the present disclosure.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. An electrochemical sensor configured to detect neurotransmitters, wherein the sensor comprises:
a substrate;
a working electrode coupled to the substrate, the working electrode includes a tellurene-based material; and
an adhesive layer.

2. The sensor of claim 1, wherein the substrate includes a polyester material.

3. The sensor of claim 2, wherein the substrate is flexible.

4. The sensor of claim 3, further comprising a polydimethylsiloxane (PDMS) layer coupled to the substrate.

5. The sensor of claim 4, wherein the PDMS layer includes microfluidic channels.

6. The sensor of claim 5, wherein the adhesive layer is permeable to sweat.

7. The sensor of claim 4, wherein the working electrode includes a metallic nanoparticle disposed on the tellurene-based material.

8. The sensor of claim 7, wherein the metallic nanoparticle is gold.

9. The sensor of claim 7, wherein the working electrode simultaneously monitors a plurality of neurotransmitters.

10. The sensor of claim 9, wherein the working electrode has a thickness of around one μm.

11. The sensor of claim 7, further comprising a reference electrode and a counter electrode.

12. The sensor of claim 11, wherein the reference electrode includes a silver/silver chloride-based material.

13. The sensor of claim 12, wherein the counter electrode includes a carbon-based material.

14. The sensor of claim 2, wherein the substrate includes a polyethylene terephthalate material.

15. The sensor of claim 13, wherein the sensor simultaneously detects dopamine and norepinephrine in real time.

16. A method of manufacturing an electrochemical sensor, the method comprising the steps of:
drop casting a tellurene crystal on a substrate, thus forming a tellurene layer;
coupling a metallic nanoparticle to the tellurene layer, thus providing a working electrode;
coupling the working electrode to an adhesive layer.

17. The method of claim 16, furthering comprising a step of coupling a silver/silver chloride-based material to the tellurene layer, thus providing a reference electrode.

18. The method of claim 17, further comprising a step of coupling a carbon-based material to the tellurene layer, thus providing a counter electrode.

19. The method of claim 16, wherein the metallic nanoparticle is gold.

20. The method of claim 18, further comprising a step of coupling a polydimethylsiloxane (PDMS) layer to the substrate before the step of coupling the working electrode to the adhesive layer.
